SEQUENCE LISTING (3 sequences)

SEQ ID NO: 1 - DNA, 18 bp, Artificial sequence
  Primer
ggccgcgtctcctttgag

SEQ ID NO: 2 - DNA, 23 bp, Artificial sequence
  Primer
cgagttgtccacagtcagcaatg

SEQ ID NO: 3 - DNA, 26 bp, Artificial sequence
  Primer
aatggcaagaccagcaagaagatcac

(19) United States
(12) Patent Application Publication
(10) Pub. No.: US 2006/0040303 A1
(43) Pub. Date: Feb. 23, 2006

(54) Reaction mixture for positioning a reaction vessel relative to a detection unit

(76) Inventor: Christian Weilke, Penzberg (DE)

Correspondence Address: Roche Diagnostics Corporation, 9115 Hague Road, PO Box 50457, Indianapolis, IN, 46250-0457, US

(21) Appl. No.: 11/203,845

(22) Filed: Aug. 15, 2005

(30) Foreign Application Priority Data
Aug. 18, 2004 (EP) EP 04019542.2

(51) Int. Cl. C12Q 1/68; C12P 19/34

(52) U.S. Cl. 435/6; 435/91.2

(57) ABSTRACT

The present invention concerns methods for measuring a nucleic acid amplification in real-time comprising (i) providing a real-time PCR instrument containing a reaction vessel holder for holding multiple reaction vessels and means for positioning the multiple reaction vessels in a reaction vessel holder relative to the detection unit, (ii) filling multiple reaction vessels with a reaction mixture containing thermostable polymerase, deoxynucleotides and buffer, at least two amplification primers, at least one fluorescently-labelled hybridization probe and a fluorescent dye component which is present in a free form during and after the entire amplification reaction, (iii) determining the position of maximum fluorescence emission of the fluorescent dye component in multiple reaction vessels as a function of their position relative to the detection unit, and (iv) performing the amplification reaction and measuring the fluorescence emission of the at least one hybridization probe during and optionally after the amplification reaction for each reaction vessel at the position of maximum fluorescence emission.

### FIELD OF THE INVENTION

The present invention concerns a system for the improved optical measurement of analytical reactions and in particular the measurement of real-time PCR amplification reactions with the aid of fluorescence measurement.

### BACKGROUND

Real-time PCR methods are nowadays among the established standard methods for analyzing nucleic acids in many molecular biological research laboratories and diagnostic institutions. Various instrument platforms from diverse manufacturers are available on the market for carrying out such methods.

The LIGHTCYCLER Instrument (Roche Diagnostics GmbH, Mannheim, Germany) is a system that has been established for several years. The LIGHTCYCLER instrument is a real-time thermocycler instrument in which capillary reaction vessels are heated or cooled with the aid of an air current. Fluorescently-labelled hybridization probes or intercalating dyes are excited with the aid of a light-emitting diode (LED), and the fluorescence is detected with the aid of a fluorimeter containing multiple photohybrids for detecting various wavelengths (WO 97/46712). The emitted light is excited and also measured with the aid of an optical path parallel to the longitudinal axis of the capillaries (FIG. 1). The excitation light is beamed into the reaction vessel through the lower end of the capillary. A large proportion of the emitted light is reflected by the capillary wall and again passes in the opposite direction through the lower end of the capillary to be detected on the photohybrids.

Due to the small diameter of the capillaries and the limited detection angle of the fluorimeter, the measuring position has to be exactly adjusted for each capillary. The capillaries are located in a rotatable carousel as the reaction vessel holder which is suitable for holding 32 capillaries. Each capillary can be tangentially positioned at a certain measuring position with the aid of a first stepping motor which rotates the carousel. A second stepper motor can radially position the fluorimeter at a certain position relative to the capillary that is to be measured in each case.

All previous instrument versions of the LIGHTCYCLER instruments determine the optimal position for the fluorescence measurement (seek) for each individual capillary at the start of an experiment. For this purpose the capillary filled with the reaction mixture in the capillary holder, i.e. for example in the LIGHTCYCLER carousel, is moved over the measuring window of the fluorimeter. In the region of the expected position it is moved further in small steps and the position of maximum fluorescence is determined. At this position the fluorimeter is moved at right angles thereto by a second stepper motor, and the position of maximum fluorescence is likewise determined. This x,y position is stored for each capillary, and this position is moved to at each subsequent measurement.

The capillaries used as reaction vessels are so thin that a high signal intensity is only measured in a very limited region. Due to variability in the shape of the capillaries and carousel such a seek has to be initially carried out for each experiment (LIGHTCYCLER Operator's Manual, Version 3.5, Oct. 2000).

For the seek the reaction mixture has to already have an adequately high background fluorescence before the PCR since otherwise it is not possible to determine a maximum of the fluorescence signal. The background fluorescence can be so low especially with the non-fluorescent quenchers that are being increasingly used for TAQMAN (Roche Molecular Systems, Inc.) probes that capillaries cannot be positioned in the LIGHTCYCLER instrument.

Hence the object of the present invention was to find a solution for correctly positioning capillaries containing a reaction mixture which have no fluorescence or only a slight fluorescence before the reaction begins.

### SUMMARY OF THE INVENTION

This object is achieved according to the invention by a method for measuring a nucleic acid amplification in real-time comprising:
(a) providing a real-time PCR instrument containing a reaction vessel holder for holding multiple reaction vessels and means for positioning the multiple reaction vessels in a reaction vessel holder relative to the detection unit,
(b) filling multiple reaction vessels with a reaction mixture containing thermostable polymerase, deoxynucleotides and buffer, at least two amplification primers, at least one fluorescently-labelled hybridization probe and one free fluorescent dye component having a constant fluorescence intensity which is independent of the amplification reaction,
(c) determining the position of maximum fluorescence emission of the fluorescent dye component in multiple reaction vessels as a function of their position relative to the detection unit, and
(d) performing amplification reactions and measuring the fluorescence emission of the at least one hybridization probe during and optionally after the amplification reaction for each reaction vessel at the position of maximum fluorescence emission from step (c).

The position of the individual reaction vessel relative to the detection unit can be changed by changing the position of the reaction vessel holder. However, the relative position can also be changed by changing the position of the detection unit. Moreover, both positions can also be changed for an exact positioning.

This method is clearly delimited from the use of a reference dye in the PCR reaction mixture as used in ABI products (U.S. Pat. No. 5,736,333, 7th Apr. 1998). In the case of ABI the reference dye (ROX) is used to normalize the signal levels of different reaction vessels in order to compensate for variances in the optical measuring unit, in the microtiter plates and in the composition and filling levels of the reagent mixtures.

The present invention also concerns a system consisting of:
a real-time PCR instrument containing means for positioning multiple reaction vessels in a reaction vessel holder relative to a detection unit and
a reaction mixture containing thermostable polymerase, deoxynucleotides, buffer and a free fluorescent dye component which does not interact with the amplification reaction.

Such systems preferably contain capillaries as reaction vessels.

Another subject matter of the invention is a reaction mixture containing thermostable polymerase, deoxynucleotides, buffer and additionally a fluorescent dye component having an emission wavelength of at least 600 nm which is independent of the amplification reaction.

Furthermore such mixtures preferably contain at least two amplification primers and at least one fluorescently-labelled hybridization probe, for example a TAQMAN probe.

Finally a subject matter of the invention is also a kit containing thermostable polymerase, deoxynucleotides and buffer and additionally a fluorescent dye component having an emission wavelength of at least 600 nm which is independent of the amplification reaction. Such kits can additionally contain specific hybridization probes such as TAQMAN probes and also amplification primers.

### DESCRIPTION OF THE FIGURES

FIG. 1: Construction of the LIGHTCYCLER instrument

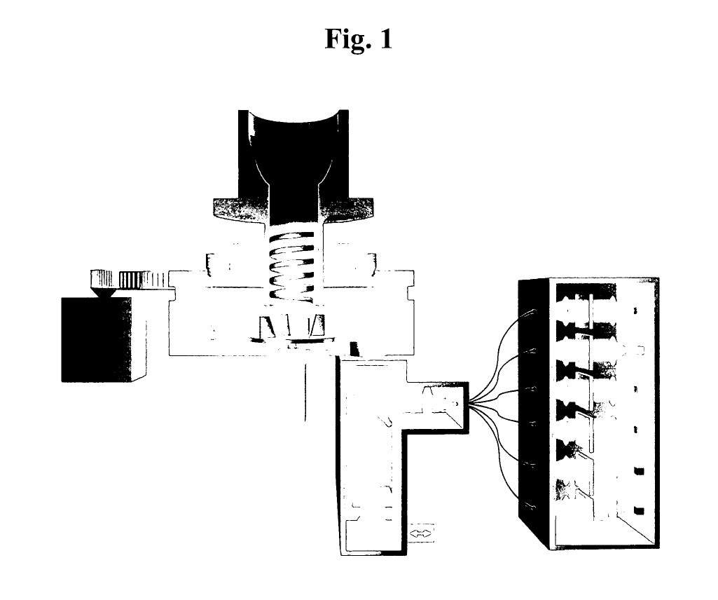

FIG. 2: Amplification curves of a cyclophilin A PCR in the presence of various JA286 concentrations.

FIG. 3: Cyclophilin A PCR with different amounts of human genomic DNA in the presence of the dye JA286

The invention is further elucidated by the following examples, publications, the sequence protocol and the figures, the protective scope of which results from the patent claims. The described methods are to be understood as examples which still describe the subject matter of the invention even after modifications

### DETAILED DESCRIPTION OF THE INVENTION

A first aspect of the present invention concerns a method for measuring a nucleic acid amplification in real-time comprising:
(a) providing a real-time PCR instrument containing a reaction vessel holder for holding multiple reaction vessels and means for positioning the multiple reaction vessels in a reaction vessel holder relative to the detection unit
(b) filling multiple reaction vessels with a reaction mixture containing thermostable polymerase, deoxynucleotides and buffer, at least two amplification primers, at least one fluorescently-labelled hybridization probe and a fluorescent dye component which is present in a free form during and after the entire amplification reaction,
(c) determining the position of maximum fluorescence emission of the fluorescent dye component in multiple reaction vessels as a function of their position relative to the detection unit, and
(d) performing amplification reactions and measuring the fluorescence emission of the at least one hybridization probe during and optionally after the amplification reaction for each reaction vessel at the position of maximum fluorescence emission from step (c).

The term “fluorescent dye component” firstly encompasses any type of free, unbound fluorescent dye where long wavelength fluorescent dyes having an emission maximum of more than 600 nm have proven to be particularly advantageous since it does not affect the measurement in the shorter wavelength detection channels. In addition the term “fluorescent dye component” also encompasses fluorescent dyes which are coupled to another chemical component provided that such molecules do not interfere with the amplification reaction and are not incorporated into the amplificate during the reaction.

Due to the fact that in the case of the LIGHTCYCLER carousel not only do the dimensions of the individual holes for the capillaries differ from one another but also the dimensions of the capillaries differ from one another within certain limits, it is necessary to position each individual capillary before each experiment. Such a procedure is essential when capillaries are used as reaction vessels since it is not possible to carry out a reliable fluorescence measurement without an exact positioning of such reaction vessels in the optical path of the detection unit i.e. of the fluorimeter.

According to the invention the means or several means for positioning multiple reaction vessels in a reaction vessel holder relative to the detection unit can be any type of finely adjustable drive. The means is preferably one or more stepper motors which can change the position of the reaction vessel holder or the position of the detection unit in very small steps.

According to the invention the detection unit can be any type of detector which is able to detect fluorescence signals. These include for example fluorimeters with one or more photohybrid diodes and also CCD cameras.

Detection Formats

The LIGHTCYCLER instrument described above can be used for various detection formats:

Intercalating DNA Binding Dyes:

In this case the respective amplification product is detected by a DNA binding dye which, on interaction with double-stranded nucleic acid, emits a corresponding fluorescence signal after excitation with light of a suitable wavelength. The dyes SYBR Green and SYBR Gold (Molecular Probes) have proven to be particularly suitable for this application. The background fluorescence of SYBR Green before the start of an amplification reaction is usually sufficient to carry out a positioning process.

FRET Hybridization Probes:

Two single-stranded hybridization probes are used simultaneously for this test format which are complementary to neighbouring sites on the same strand of the amplified target nucleic acid. Both probes are labelled with different fluorophores which differ in their absorbance and emission wavelength. When the short wavelength fluorophore is excited without the target nucleic acid, the fluorescence of this fluorophore can be measured almost exclusively. When both probes hybridize to the amplified target nucleic acid, the two fluorophores come into such close proximity that the excited fluorophore can transfer the absorbed energy to the second fluorophore according to the principle of fluorescence energy transfer and its emission can be measured in the long wavelength channel (WO 97/46707). In this case fluorescein as a so-called donor component is for example excited with light of a suitable wavelength. When it comes into spatial proximity to a suitable acceptor component such as certain rhodamine derivatives, resonance energy is then transferred to the acceptor component such that the acceptor fluorophore emits light of a higher emission wavelength. Also in this format the emission of the FRET donor is sufficient to carry out a positioning process.

TAQMAN Hybridization Probes:

A single-stranded hybridization probe is labelled with 2 components. When the first component is excited with light of a suitable wavelength, the absorbed energy is transferred to the second component, the so-called quencher according to the principle of fluorescence resonance energy transfer such that the radiation emitted by the first component is almost completely suppressed. During the annealing step of the PCR reaction, the hybridization probe binds to the target DNA and is cleaved by the 5′-3′ exonuclease activity of the Taq polymerase during primer elongation. As a result the excited fluorescent component and the quencher are spatially separated so that a fluorescence emission of the first component can be measured at an appropriate wavelength (U.S. Pat. No. 5,210,015, U.S. Pat. No. 5,487,972, U.S. Pat. No. 5,804,375, U.S. Pat. No. 6,214,979).

Due to the fact that such hybridization probes radiate almost no fluorescence emission with non-fluorescent quenchers in the unbound intact state even when the first component is excited, it is essential for this format that detectable dye components that can be used to position the reaction vessels in the LIGHTCYCLER are already added to the reaction mixture before the amplification.

Addition of a Fluorescent Dye Component

Hence in order to reliably position the LIGHTCYCLER capillaries especially in the case of reaction mixtures having a low background fluorescence such as TAQMAN probes with non-fluorescent quenchers, a passive fluorescent dye which does not influence the PCR and does not interfere with the optical detection (seek dye) is added according to the invention to the reaction mixture. In other words according to the invention the reaction mixture contains a fluorescent dye component which is present in a free form during and after the entire amplification reaction. This means that the dye component is not covalently incorporated into the amplification products in the form of labelled primers or labelled dNTPs during their amplification and moreover also does not have a binding affinity for a double-stranded DNA amplification product as is for example the case for SYBR Green.

The emission maximum of this passive seek dye is preferably in the long wavelength range of more than 600 nm and particularly preferably in the longest wavelength detection channel of the six-channel LIGHTCYCLER 2.0 instrument. This has no effect on detection in the shorter wavelength channels 530-640 nm which are used almost exclusively for LIGHTCYCLER TAQMAN assays due to the commercially available and suitable dyes. Only a constant elevation of the background signal is visible in the channels 705 and 670 nm that usually does not interfere with the measurement of changes in the signal level. The seek dye has a sufficiently high stability towards temperature and light stress so that the properties do not change during the PCR. In this connection the use of JA286 (EP 0 747 447, example 1) has proven to be particularly advantageous.

The present invention also concerns a system consisting of:
a real-time PCR instrument containing means for positioning multiple reaction vessels in a reaction vessel holder relative to a detection unit and
a reaction mixture containing thermostable polymers, deoxynucleotides, buffer and a fluorescent dye component where the fluorescent dye component is present in a completely free form during and after an entire amplification reaction.

The system according to the invention preferably contains capillaries as reaction vessels.

The reaction mixture also already preferably contains at least two amplification probes and at least one hybridization probe such as a TAQMAN hybridization probe.

In addition the system preferably contains a fluorescent dye having an emission maximum of more than 600 nm as the fluorescent dye component, for example the fluorescent dye JA 286 (EP 0 747 447, Example 1).

Positioning Process

The positioning process (seek process) can be carried out in the LIGHTCYCLER for an individual capillary as follows:

Before each experiment (run) the optimal measuring positions have to be determined in a radial and tangential direction for all capillaries with the reaction mixtures contained therein. These positions always apply to an entire run and have to be redetermined at each new start of a protocol.

The closed chamber is heated to 30° C. or another specified temperature for the run. After the temperature is reached a home run is carried out in which the light barriers for the photometer and rotor are moved to as a reference position. Afterwards the position of the maximum fluorescence signal is determined for each sample. The measurement is carried out in all detection channels, the channel with the highest signal is evaluated.

The target position for capillary 1 is stored as CarOffset and RadOffset in a memory location referred to as EEPROM. A window in which the seek for capillary 1 is carried out is spanned around this position. The measurement is carried out along 4 crossing paths (“#”) and hence this process is referred to as cross-seek. For each of the following capillaries the sample carousel is rotated further by 1/32 of the circumference from each determined position and a cross-seek is again carried out.

The cross-seek is carried out in 4 phases:
Phase 1=carousel

The maximum signal is determined and stored by rotating the sample carousel in small steps within the defined window around the start-up position.
Phase 2=photometer

At the maximum rotor position of phase 1 the photometer position of the maximum signal is determined within the defined window and stored.
Phase 3=carousel

At the maximum photometer position of phase 2 the carousel position with the maximum signal is determined within the new window around the carousel position from phase 1 and stored.
Phase 4=photometer

At the maximum carousel position of phase 3 the photometer position with the maximum signal is determined within the new window around the photometer position from phase 2 and stored.

This capillary is only declared to be found when the maximum fluorescence exceeds a specified minimum value. If the signal is too low this capillary is regarded as not found. This avoids the needless positioning or incorrect evaluation of positions without reaction mixture. If the capillaries are regarded as found, the determined coordinates of the rotor and photometer from phase 4 are stored and are moved to in each case when the capillaries are measured in the following RUN.

Mixtures and Kits

In another aspect the present reaction mixture contains thermostable polymerase, deoxynucleotides, buffer and additionally a fluorescent dye component which is present in the a form during and after the entire amplification reaction. It is preferably a dye having an emission wavelength of more than 600 nm. The fluorescent dye JA286 is particularly preferred.

Such mixtures can be prepared as master mixes for multiple reactions. After appropriate aliquoting such mixtures can be additionally provided with at least two specific amplification primers and with at least one fluorescently-labelled hybridization probe such as a TAQMAN probe before final use in an amplification reaction.

The present invention additionally concerns kits containing thermostable polymerase, deoxynucleotides and buffer and additionally a fluorescent dye component having an emission wavelength of at least 600 nm which is present in a completely free form during and after the entire amplification reaction. It is preferably also a dye having an emission wavelength of more than 600 nm. The fluorescent dye JA286 is particularly preferred.

In this context one distinguishes between two types of kits:

Generic kits contain no other components and can thus be used for any type of real-time PCR independent of the detection format and independent of the target sequence to be amplified. Hence in this case the end user must additionally add primers and hybridization probes according to his requirements on an individual basis.

Parameter-specific kits usually additionally contain at least two amplification primers and at least one fluorescently-labelled hybridization probe, preferably a TAQMAN probe.

ASR's (analyte specific reagents) are a special case. Such kits contain at least two amplification primers, at least one fluorescently-labelled hybridization probe and according to the invention a fluorescent dye component.

Description of the Sequence Protocol

### EXAMPLE 1

Starting with 300 pg human genomic DNA, a 442 bp fragment of the cyclophilin A gene was amplified in a LIGHTCYCLER experiment using the following primers according to a standard thermocycling protocol in the presence of various amounts of the fluorescent dye JA286 (EP 0 747 447, Example 1):

| | |
|---|---|
| Primer: | |
| 5′-GGC CGC GTC TCC TTT GAG | (SEQ ID NO:1) |
| 5′-CGA GTT GTC CAC AGT CAG CAA TG | (SEQ ID NO:2) |

The amplification was carried out under the following conditions:

| | | | Target | | Trans. | | Step | Step | |
|---|---|---|---|---|---|---|---|---|---|
| | | Analysis | Temp. | Hold | Rate | Sec. | Size | Delay | Acquisition |
| Program | Cycles | Mode | [° C.] | [hh:mm:ss] | [° C./s] | Target | [° C.] | [Cycl.] | Mode |
| Heating | 1 | None | 95 | 00:10:00 | 20 | 0 | 0 | 0 | None |
| Cycling | 45 | Quantif. | 95 | 00:00:10 | 20 | 0 | 0 | 0 | None |
| | | | 55 | 00:00:30 | 20 | 0 | 0 | 0 | None |
| | | | 72 | 00:00:01 | 20 | 0 | 0 | 0 | Single |
| Cooling | 1 | | 40 | 00:00:30 | 20 | 0 | 0 | 0 | None |

The amplification product was detected with the aid of the following TAQMAN probe in the real-time mode:

Probe:

(SEQ ID NO:3)

FAM-5′-AAT GGC AAG ACC AGC AAG AAG ATC AC-TAMRA

The capillaries were exactly positioned even when only 0.5 μM JA286 was added.

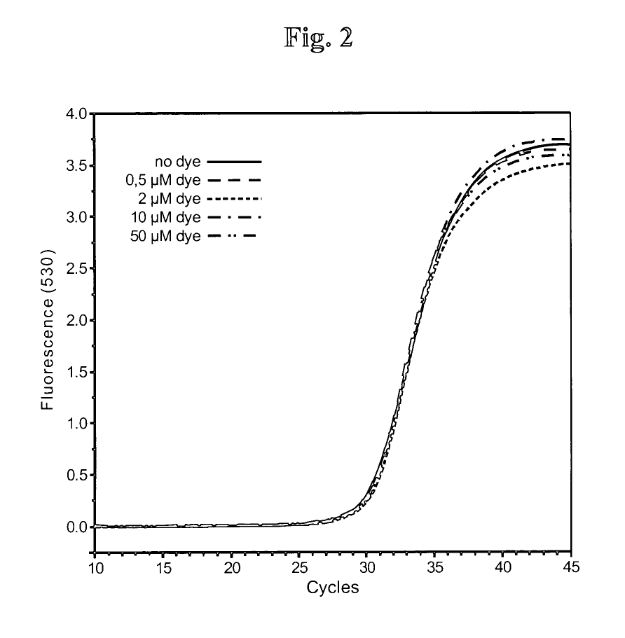

As shown in FIG. 2 no inhibition of the amplification reaction was found even when up to 50 mm JA286 was added.

### EXAMPLE 2

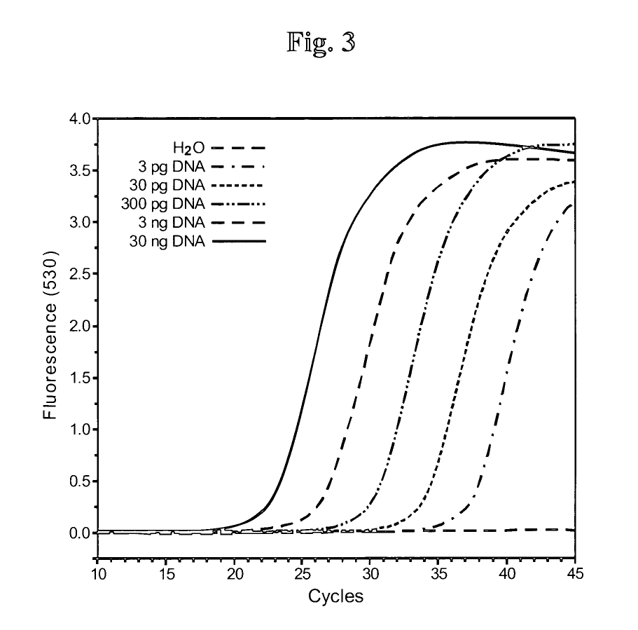

In a further experiment like example 1, various amounts (3 pg, 30 pg, 300 pg, 3 ng, 30 ng) of genomic template DNA was used to amplify the cyclophilin A gene while in each case adding a constant amount of 0.5 μM JA286 dye. As shown in FIG. 3 the presence of the additional dye did not result in a falsification of the quantitative result neither at low nor at high template concentrations.

What is claimed is:

1. A method for measuring a nucleic acid amplification reaction in real-time comprising:
(a) providing a real-time PCR instrument containing a reaction vessel holder for holding multiple reaction vessels and a means for positioning the multiple reaction vessels in a reaction vessel holder relative to a detection unit,
(b) filling the multiple reaction vessels with a reaction mixture containing thermostable polymerase, deoxynucleotides, and a buffer, at least two amplification primers, at least one fluorescently-labelled hybridization probe, and a fluorescent dye component which is present in a free form during and after the amplification reaction,
(c) determining the position of maximum fluorescence emission of the fluorescent dye component in the multiple reaction vessels as a function of their position relative to the detection unit, and
(d) performing amplification reactions and measuring the fluorescence emission of the hybridization probe during and after the amplification reaction for each reaction vessel at the position of maximum fluorescence emission from step (c).

2. The method of claim 1 wherein the position of the reaction vessel holder is changed.

3. The method of claim 1 wherein the position of the detection unit is changed.

4. A system consisting of:
(a) a real-time PCR instrument containing a means for positioning multiple reaction vessels in a reaction vessel holder relative to a detection unit and
(b) a reaction mixture containing a thermostable polymerase, deoxynucleotides, buffer, and a fluorescent dye component, wherein the fluorescent dye component is present in a completely free form during and after an amplification reaction.

5. The system of claim 4 wherein the reaction vessels are capillaries.

6. A mixture for use in an amplification reaction, the mixture containing a thermostable polymerase, deoxynucleotides, a buffer, and a fluorescent dye component having an emission wavelength of at least 600 nm, wherein the dye component is selected such that it is present in a free form during and after the amplification reaction.

7. The reaction mixture of claim 6 additionally containing at least two amplification primers and at least one fluorescently-labelled hybridization probe.

8. The reaction mixture of claim 7 wherein the fluorescently-labelled hybridization probe is a TAQMAN probe.

9. A kit for use in an amplification reaction, the kit containing a thermostable polymerase, deoxynucleotides, a buffer, and a fluorescent dye component having an emission wavelength of at least 600 nm, wherein the dye component is selected such that it is present in a completely free form during and after the amplification reaction.

10. The kit of claim 9 additionally containing a TAQMAN probe.
